SEQUENCE LISTING (3 sequences)

SEQ ID NO: 1 - protein, 25 aa, Bos taurus
FKCRRWQWRMKKLGAPSITCVRRAF

SEQ ID NO: 2 - DNA, 22 bp, Artificial Sequence
  synthetic oligonucleotide
gcctcttctcgttcctcatcac

SEQ ID NO: 3 - DNA, 19 bp, Artificial Sequence
  synthetic oligonucleotide
agcagcatcaccgccattg

(19) United States
(12) Patent Application Publication
(10) Pub. No.: US 2024/0115667 A1
(43) Pub. Date: Apr. 11, 2024

(54) DIPHENHYDRAMINE AND LACTOFERRIN FOR PREVENTION AND TREATMENT OF COVID-19

(71) Applicant: University of Florida Research Foundation, Incorporated, Gainesville, FL (US)

(72) Inventors: David A. OSTROV, Gainesville, FL (US); Michael NORRIS, Gainesville, FL (US)

(21) Appl. No.: 18/257,237

(22) PCT Filed: Dec. 15, 2021
(86) PCT No.: PCT/US2021/072932

§ 371 Date: Jun. 13, 2023

Related U.S. Application Data
(60) Provisional application No. 63/126,082, filed on Dec. 16, 2020.

(51) Int. Cl. A61K 38/40; A61K 31/137; A61K 31/138; A61K 31/167; A61K 31/426; A61K 31/451; A61K 31/485; A61K 31/625; A61P 31/14

(52) U.S. Cl. CPC A61K 38/40; A61K 31/137; A61K 31/138; A61K 31/167; A61K 31/426; A61K 31/451; A61K 31/485; A61K 31/625; A61P 31/14

(57) ABSTRACT

Compositions and formulations comprising diphenhydramine and lactoferrin for preventing or treating infection by a SARS-CoV-related betacoronavirus are described. Methods of using the compositions and formulations are also described. The methods include administering to a patient at risk of being infected by a SARS-CoV-related betacoronavirus or suffering from SARS-CoV-related betacoronavirus-related illness a composition or formulation comprising diphenhydramine and lactoferrin.

### CROSS-REFERENCE TO RELATED APPLICATIONS

This application is a 35 U.S.C. § 371 National Stage of International Application No. PCT/US2021/072932, filed Dec. 15, 2021, which claims the benefit of U.S. Provisional Application No. 63/126,082, filed 16 Dec. 2020, each of which is incorporated herein by reference.

### SEQUENCE LISTING

The Sequence Listing written in file T18371_SeqListing.txt is 1 kilobyte in size, was created 14 Dec. 2021, and is hereby incorporated by reference.

### INTRODUCTION

COVID-19 is a global health crisis caused by the novel coronavirus SARS-CoV-2. In severe cases, SARS-CoV-2 infection causes respiratory failure and death. SARS-CoV-2 gains access to airway cells through binding to the angiotensin converting enzyme 2 (ACE2).

The ACE2 gene encodes the angiotensin-converting enzyme-2, which has been proved to be the receptor for both the SARS-coronavirus (SARS-CoV) and the human respiratory coronavirus NL63. Recent studies and analyses indicate that ACE2 could be the host receptor for the novel coronavirus 2019-nCoV/SARS-CoV-2.

There is an urgent need to identify therapies that prevent SARS-CoV-2 infection and improve the outcome of COVID-19 patients.

### SUMMARY

Described are combinations and formulations comprising diphenhydramine and lactoferrin for use in the prevention and/or treatment of COVID-19 and other SARS-CoV-related betacoronavirus diseases. The described combinations and formulations containing diphenhydramine and lactoferrin are useful as antiviral therapeutics in preventing and/or treating infection caused by SARS-CoV-related betacoronaviruses. In some embodiments, the SARS-CoV-related betacoronavirus is SARS-CoV or SARS-CoV-2. An infection caused by a SARS-CoV-related betacoronavirus can be, but is not limited to, COVID-19. In some embodiments, the described combinations and formulations can be used to inhibit SARS-CoV-related betacoronaviruses replication and/or infection.

The described combinations and formulations can be used to prevent or treat SARS-CoV-2 infection in a subject. In some embodiments, the described combinations and formulations are administered to a subject at risk of infection by SARS-CoV-2. In some embodiments, the described combinations and formulations are administered to a subject that has tested positive for SARS-CoV-2. In some embodiments, the described combinations and formulations are administered to a subject that has been exposed to SARS-CoV-2. In some embodiments, the described combinations and formulations are administered to a subject suspected of having been exposed to SARS-CoV-2. In some embodiments, the described combinations and formulations are administered to a subject at risk of being exposed to SARS-CoV-2. In some embodiments, the described combinations and formulations are administered to a subject suffering from or diagnosed with COVID-19. In some embodiments, the described combinations and formulations are administered to a subject to treat acute lung injury in a subject suffering from coronavirus infection, such as COVID-19.

The described combinations and formulations can be used to prevent or treat SARS-CoV-related betacoronavirus infection in a subject. In some embodiments, the described combinations and formulations are administered to a subject at risk of infection by a SARS-CoV-related betacoronavirus. In some embodiments, the described combinations and formulations are administered to a subject that has tested positive for a SARS-CoV-related betacoronavirus. In some embodiments, the described combinations and formulations are administered to a subject that has been exposed to a SARS-CoV-related betacoronavirus. In some embodiments, the described combinations and formulations are administered to a subject suspected of having been exposed to a SARS-CoV-related betacoronavirus. In some embodiments, the described combinations and formulations are administered to a subject at risk of being exposed to a SARS-CoV-related betacoronavirus. In some embodiments, the described combinations and formulations are administered to a subject suffering from or diagnosed with a SARS-CoV-related betacoronavirus illness.

In some embodiments, the diphenhydramine is a diphenhydrarnine salt. The diphenhydramine salt can be, but is not limited to, diphenhydramine HCl or diphenhydramine citrate.

The lactoferrin can be, but is not limited to, unsaturated iron lactoferrin, hololactoferrin, recombinant lactoferrin or a fragment of lactoferrin having antiviral activity. Recombinant lactoferrin can be made from a plant, such a rice, from a microorganism, such as yeast or bacteria, or from mammalian or insect cells grown in culture. The lactoferrin can be, but is not limited to, human lactoferrin or bovine lactoferrin. The lactoferrin can be derived from or obtained from milk or colostrum.

The combinations and formulations can be formulated for oral administration, parenteral administration, IV administration, injection, or inhalation (e.g., nasal delivery).

The combinations and formulations can be formulated or manufactured as a solid, a powder (e.g., a lyophilized powder), a tablet (e.g., a pill), a capsule, or a liquid. In some embodiments, the combinations and formulations can be formulated for nasal delivery.

In some embodiments, the combinations and formulations further contain a pain reliever. In some embodiments, the combinations and formulations further contain a nasal decongestant. In some embodiments, the combinations and formulations further contain a pain reliever and a nasal decongestant.

In some embodiments, pharmaceutical compositions for treating or preventing a SARS-CoV-related betacoronavirus disease, such as COVID-19, are described. The pharmaceutical compositions comprise a therapeutically effective amount of an H1 receptor blocking antihistamine and a therapeutically effective amount of lactoferrin. The H1 receptor blocking antihistamine can be selected from the group consisting of: diphenhydramine, hydroxyzine, cetirizine, azelastine, loratadine, levocetirizine, brompheniramine, fexofenadine, and chlorpheniramine. In some embodiments, the pharmaceutical compositions further comprise one or more of: a H2 receptor blocking antihistamine, a non-steroidal anti-inflammatory drug (NSAID), a cough suppressant, decongestant, and an anti-nausea or anti-diarrhea medication. In some embodiments, the pharmaceutical compositions further comprise each of: a H2 receptor blocking antihistamine, a non-steroidal anti-inflammatory drug (NSAID), a cough suppressant, decongestant, and an anti-nausea or anti-diarrhea medication. The H2 receptor blocking antihistamine can be, but is not limited to, famotidine. The NSAID can be, but is not limited to, acetaminophen. The cough suppressant can be, but is not limited to, dextromethorphan. The decongestant can be, but is not limited to, phenylephrine. The anti-nausea or anti-diarrhea medication can be, but is not limited to, bismuth subsalicylate or loperamide.

In some embodiments, the diphenhydramine and lactoferrin are formulated with one or more adjuvants, carriers, excipients, or a combination thereof.

### BRIEF DESCRIPTION OF THE DRAWINGS

FIG. 1. Model for antiviral mechanisms mediated by specific antihistamines. Diphenhydramine and related antihistamines have the potential to inhibit SARS-CoV-2 entry (by binding ACE2), and virus replication (by binding the sigma-1 receptor). Diphenhydramine (star) exhibits off-target inhibitory ACE2 activity by forming intermolecular interactions with the active site, inducing a conformational change from the open conformation to the closed conformation. The conformational change shifts the position of ACE2 at positions that contact the SARS-CoV-2 spike glycoprotein receptor binding domain (RBD), which results in a decrease in intermolecular interactions at the ACE2/RBD interface. The sigma-1 receptor is a membrane bound chaperone highjacked by SARS-CoV-2 to link the replicase/transcriptase complex to the endoplasmic reticulum by binding directly to nonstructural protein NSP6. NSP6 forms a complex with NSP3 and NSP4. Diphenhydramine binds the sigma-1 receptor, potentially interfering with the virus life cycle by blocking protein-protein interactions with NSP6.

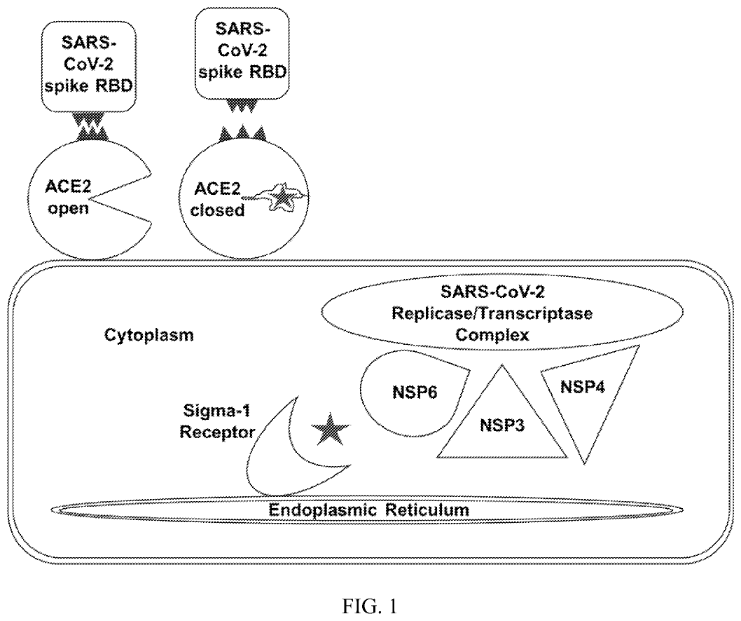

FIG. 2. Graph illustrating effectiveness of diphenhydramine (DPH), unsaturated iron human lactoferrin (hLNF milk), and recombinant human lactoferrin (rLFN) alone and in combination in reducing cytotoxicity of SARS-CoV-2 virus. Control samples (VEH) did not receive diphenhydramine or lactoferrin. Each data point was done in triplicate.

FIG. 3. Checkerboard plot illustrating inhibition of SARS-CoV-2 induced cytotoxicity in the presence of varying concentration of diphenhydramine and unsaturated iron lactoferrin (hLF).

FIG. 4. Heat map illustrating inhibition of SARS-CoV-2 induced cytotoxicity for varying concentrations of diphenhydramine and unsaturated iron lactoferrin, alone or in combination Inhibition of SARS-CoV-2 induced cytotoxicity was amplified when the two drugs were combined (100 μg/mL corresponds to 32 μM lactoferrin, 10 μg/mL corresponds to 35 μM diphenhydramine).

FIG. 5. Effective concentration 50 (EC₅₀) curves illustrating the dose dependent inhibition of the SARS-CoV-2 induced cytotoxicity of diphenhydramine. LFN concentrations in the figure legend are in order of start of the curves at the y axis (highest to lowest).

FIG. 6A-B. Graphs illustrating anti-SARS-CoV-2 activity by diphenhydramine. A), Vero E6 cells were treated with diphenhydramine (DPH) at various concentrations without (black bars) or with SARS-CoV-2 at MOI 0.2 (gray bars) and cytotoxicity was measured by LDH release. B), The EC50 (white circles) and CC50 (black circles) curves were determined by non-linear regression. The EC50 of diphenhydramine alone was 122 μg/ml.

FIG. 6C-D. Graphs illustrating anti-SARS-CoV-2 activity by diphenhydramine and lactoferrin. (C) Vero E6 cells were treated with diphenhydramine at various concentrations and lactoferrin (LFN) at 400 μg/ml without (black bars) or with SARS-CoV-2 at MOI 0.2 (gray bars) and cytotoxicity was measured by LDH release. D), The EC50 (white circles) and CC50 (black circles) curves were determined by non-linear regression. The EC50 of diphenhydramine with 400 μg/ml of lactoferrin was 54.25 μg/ml. D), The EC50 curves of DPH (white circles), LFN (black diamonds), and DPH+LFN (black squares) are shown on the same graph to compare effect of LFN on DPH EC50.

FIG. 6E-F. Graphs illustrating anti-SARS-CoV-2 activity by diphenhydramine and lactoferrin. Combinations of diphenhydramine and lactoferrin exhibited synergy against SARS-CoV-2. (E) EC50 curves illustrating inhibition of SARS-CoV-2 mediated cytotoxicity by diphenhydramine (DPH) is enhanced in the presence of lactoferrin (LFN). (F) Measurement of viral genome equivalents by RT-qPCR of the SARS-CoV-2 N-protein gene demonstrate the ability of DPH+LFN to inhibit replication by almost 3-logs. *, p≤0.05; **, p≤0.01; ***, p≤0.001; ****, p<0.0001; ns, not significant.

FIG. 7A. Micrographs illustrating sensitivity of ACE2-transfected human lung epithelial cells to SARS-CoV-2 infection. NCI-H23 (parental untransduced cells), NCI-H23ACE2 pool (lentivirus transformed cells uncloned), and NCI-H23ACE2 (clone A2) were infected with SARS-CoV-2 and cytopathic effects was observed 3 dpi. CPE is defined by cell rounding and detachment from the monolayer. The scale bar is equivalent to 100 μm.

FIG. 7B-C. Graphs illustrating anti-SARS-CoV-2 activity by diphenhydramine and lactoferrin in human lung epithelial cells. (B) TCID50 experiments were performed in biological duplicate three times after infecting cells at an MOI of 0.01 for 72 h. SARS-CoV-2 infection of the human lung epithelial cell line H23 was dependent on heterologous expression of the human ACE2 receptor. (C) Diphenhydramine and diphenhydramine with lactoferrin significantly reduced infectious SARS-CoV-2 particle release from H23-hACE2 cells by −1-log compared to untreated H23-hACE2 cells. Data are from TCID50s carried out in technical triplicate. *, p≤0.05; ****, p≤0.0001; ns, not significant.

### DETAILED DESCRIPTION

Before describing the present teachings in detail, it is to be understood that the disclosure is not limited to specific compositions or process steps, as such may vary. It should be noted that, as used in this specification and the appended claims, the singular form “a,” “an,” and “the” include plural references unless the context clearly dictates otherwise. Thus, for example, reference to “a drug” includes a plurality of drugs and the like. The conjunction “or” is to be interpreted in the inclusive sense, i.e., as equivalent to “and/or,” unless the inclusive sense would be unreasonable in the context.

In general, the term “about” indicates insubstantial variation in a quantity of a component of a composition not having any significant effect on the activity or stability of the composition. When the specification discloses a specific value for a parameter, the specification should be understood as alternatively disclosing the parameter at “about” that value. All ranges are to be interpreted as encompassing the endpoints in the absence of express exclusions such as “not including the endpoints”; thus, for example, “within 10-15” or “from 10 to 15” includes the values 10 and 15. Also, the use of “comprise,” “comprises,” “comprising,” “contain,” “contains,” “containing,” “include,” “includes,” and “including” are not intended to be limiting. It is to be understood that both the foregoing general description and detailed description are exemplary and explanatory only and are not restrictive of the teachings. To the extent that any material incorporated by reference is inconsistent with the express content of this disclosure, the express content controls.

Unless specifically noted, embodiments in the specification that recite “comprising” various components are also contemplated as “consisting of” or “consisting essentially of” the recited components. Embodiments in the specification that recite “consisting essentially of” various components are also contemplated as “consisting of”. “Consisting essentially of” means that additional component(s), composition(s) or method step(s) that do not materially change the basic and novel characteristics of the compositions and methods described herein may be included in those compositions or methods.

A “SARS-CoV-related betacoronavirus” is a virus that is considered highly similar to or phylogenetically similar to 2003 SARS-CoV or 2019 SARS-CoV-2. A SARS-CoV-related betacoronaviruses can be a betacoronavirus in Lineage B, subgenus Sarbecovirus, or Lineage D, subgenus Nobecovirus. Betacoronaviruses in Lineage A, subgenus Embecovirus (including common human coronaviruses OC43 and HKU1) and Lineage C, subgenus Merbecovirus (including Middle East respiratory syndrome coronavirus) are not considered SARS-CoV-related betacoronaviruses.

A “homologous” sequence (e.g., nucleic acid sequence or amino acid sequence) refers to a sequence that is either identical or substantially similar to a known reference sequence, such that it is, for example, at least 75%, at least 80%, at least 85%, at least 90%, at least 95%, at least 96%, at least 97%, at least 98%, at least 99%, or 100% identical to the known reference sequence. Sequence identity can be determined by aligning sequences using algorithms, such as BESTFIT, FASTA, and TFASTA in the Wisconsin Genetics Software Package Release 7.0, Genetics Computer Group, 575 Science Dr., Madison, Wis.), using default gap parameters, or by inspection, and the best alignment (i.e., resulting in the highest percentage of sequence similarity over a comparison window). Percentage of sequence identity is calculated by comparing two optimally aligned sequences over a window of comparison, determining the number of positions at which the identical residues occurs in both sequences to yield the number of matched positions, dividing the number of matched positions by the total number of matched and mismatched positions not counting gaps in the window of comparison (i.e., the window size), and multiplying the result by 100 to yield the percentage of sequence identity. Unless otherwise indicated the window of comparison between two sequences is defined by the entire length of the shorter of the two sequences.

Peptide variants and derivatives are well understood to those of skill in the art and can involve amino acid sequence modifications. An amino acid sequence modification can be a substitution, insertion, or deletion. Insertions include amino and/or carboxyl terminal additions as well as intrasequence insertions of single or multiple amino acid residues. Deletions include the removal of one or more amino acid residues from the peptide sequence. Substitutions include substitution of an amino acid residue at a given position in the amino acid sequence with a different amino acid. Insertions, deletions, and substitutions can occur at a single position or multiple positions. Insertions, deletions, and substitutions can occur at adjacent positions and/or non-adjacent positions. In some embodiments the one or more of the substitutions is a conservative amino acid substitution. Substitutions, deletions, insertions, or any combination thereof may be combined to arrive at a final S-protein polypeptide. For a peptide differing by 0, 1, 2, or 3 amino acids from a reference sequence, the peptide can have substitutions, insertions, or deletions of 0, 1, 2, or 3 amino acids in any combination or order.

An “active ingredient” is any component of a drug product intended to furnish pharmacological activity or other direct effect in the diagnosis, cure, mitigation, treatment, or prevention of disease, or to affect the structure or any function of the body of humans or other animals. Active ingredients include those components of the product that may undergo chemical change during the manufacture of the drug product and be present in the drug product in a modified form intended to furnish the specified activity or effect. A dosage form for a pharmaceutical contains the active pharmaceutical ingredient, which is the drug substance itself, and excipients, which are the ingredients of the tablet, or the liquid in which the active agent is suspended, or other material that is pharmaceutically inert. During formulation development, the excipients can be selected so that the active ingredient can reach the target site in the body at the desired rate and extent.

A “pharmacologically effective amount,” “therapeutically effective amount,” or simply “effective amount” refers to that amount (dose) of a described active pharmaceutical ingredient or pharmaceutical composition to produce the intended pharmacological, therapeutic, or preventive result. An “effective amount” can also refer to the amount of, for example an excipient, in a pharmaceutical composition that is sufficient to achieve the desired property of the composition. An effective amount can be administered in one or more administrations, applications, or dosages.

As used herein, “dose,” “unit dose,” or “dosage” can refer to physically discrete units suitable for use in a subject, each unit containing a predetermined quantity of active pharmaceutical ingredient and/or a pharmaceutical composition thereof calculated to produce the desired response or responses in association with its administration.

The terms “treat,” “treatment,” and the like, mean the methods or steps taken to provide relief from or alleviation of the number, severity, and/or frequency of one or more symptoms of a disease or condition in a subject. Treating generally refers to obtaining a desired pharmacological and/or physiological effect. The effect can be, but does not necessarily have to be, prophylactic in terms of preventing or partially preventing a disease, symptom or condition thereof. The effect can be therapeutic in terms of a partial or complete cure of a disease, condition, symptom or adverse effect attributed to the disease, disorder, or condition. The term treatment can include: (a) preventing the disease from occurring in a subject who may be predisposed to the disease but has not yet been diagnosed as having it; (b) inhibiting the disease, i.e., arresting its development; and (c) relieving the disease, i.e., mitigating or ameliorating the disease and/or its symptoms or conditions. Treating can refer to both therapeutic treatment alone, prophylactic treatment alone, or both therapeutic and prophylactic treatment. Those in need of treatment (subjects in need thereof) can include those already with coronavirus infection that or those in which infection is to be prevented. Treating can include inhibiting the disease, disorder or condition, e.g., impeding its progress; and relieving the disease, disorder, or condition, e.g., causing regression of the disease, disorder and/or condition. Treating the disease, disorder, or condition can include ameliorating at least one symptom of the particular disease, disorder, or condition, even if the underlying pathophysiology is not affected, e.g., such as treating the inflammation without preventing viral replication.

“Diphenhydramine” is an over the counter antihistamine with a long safety history. Diphenhydramine is readily available and has a favorable safety profile. In silico molecular docking suggests that diphenhydramine has the potential to interact with the Sigma-1 receptor and inhibit or reduce SARS-CoV-related betacoronavirus infection.

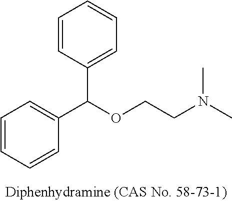 [SMILES: CN(C)CCOC(c1ccccc1)c1ccccc1]

“Lactoferrin” is an iron-binding protein present in colostrum, milk, external secretions, and polymorphonuclear leukocytes. Lactoferrin is an ˜80 kDa glycosylated protein of about 700 amino acids (711 amino acids for human lactoferrin and 689 amino acids for bovine lactoferrin) with high homology among species (orthologs). Lactoferrin functions in non-immune protection. Lactoferrin has been shown to be involved in several physiological and protective functions, including regulation of iron absorption, and antioxidant, anticancer, anti-inflammatory and antimicrobial activities. Orally administered lactoferrin has been shown to exhibit antimicrobial activity and immunomodulatory activity, including anti-inflammatory activity. Antimicrobial activity is believed to be due to iron deprivation and/or interaction with microbial cells. In addition to full length lactoferrin, three lactoferrin-derived peptides have been identified that also have antimicrobial (including antiviral) activity. These three peptides are Lf(1-11), Lactoferricin (Lfcin) and lactoferrampin (Lfampin). Lf(1-11) includes the first eleven amino acid residues of lactoferrin and is highly cationic in nature. It has been shown that Lf(1-11) interacts with the membrane of several bacteria. Lfcin is an amphipathic, cationic peptide generated by pepsin-mediated digestion of Lactoferrin (e.g., amino acid residues 17-41, FKCRRWQWRMKKLGAPSITCVRRAF (SEQ ID NO: 1), for bovine lactoferrin). Lfampin comprises residues 268-284 in the N1 domain of Lactoferrin. Lactoferrin has been shown to display antiviral activity against both enveloped and naked viruses, including Cytomegalovirus (CMV), Herpes simplex virus (HSV), Human immunodeficiency virus (HIV), Human hepatitis C (HCV) and human hepatitis B (HBV) viruses.

Each lactoferrin molecule can bind two ferric irons (Fe 3+), i.e., when saturated, each lactoferrin binds two iron ions. Lactoferrin from milk typically contains 10-30% iron saturation. Unsaturated lactoferrin, also termed reduced iron lactoferrin or apolactoferrin, is lactoferrin that has been depleted of iron. In some embodiments, unsaturated lactoferrin is less than 10% iron saturated. In some embodiments, unsaturated lactoferrin is less than 9% iron saturated, less than 8% iron saturated, less than 7% iron saturated, less than 6% iron saturated, less than 5% iron saturated, less than 4% iron saturated, less than 3% iron saturated, less than 2% iron saturated, or less than 1% iron saturated. In some embodiments, unsaturated lactoferrin is less than 0.5% iron saturated, or less than 0.15% iron saturated.

Described herein are combinations and formulations comprising diphenhydramine and lactoferrin. The described combinations and formulations can be used in methods for therapeutic treatment and/or prevention of symptoms and diseases associated of SARS-CoV-related betacoronavirus infection. Such methods comprise administration of the combinations and formulations as described herein to a subject, e.g., a human or animal subject.

In some embodiments, the described combinations and formulations can be administered to a subject to decrease SARS-CoV-related betacoronavirus disease burden. In some embodiments, the described combinations and formulations can be administered to a subject to decrease SARS-CoV-related betacoronavirus viral transmission. In some embodiments, the described combinations and formulations can be administered to a subject to inhibit SARS-CoV-related betacoronavirus infection, decrease the likelihood of infection, decrease the severity of infection, and/or decrease the duration of infection. In some embodiments, the described combinations and formulations can be administered to a subject infected with SARS-CoV-related betacoronavirus to decrease viral load in the subject. In some embodiments, the described combinations and formulations can be administered to a subject to inhibit SARS-CoV-related betacoronavirus entry into host cells. The SARS-CoV-related betacoronavirus can be SARS-CoV-2. In some embodiments, the described combinations and formulations can be administered to a subject to reduce the likelihood the subject will require hospitalization due to coronavirus infection.

Described are methods of decreasing viral load, decreasing disease burden, decreasing viral transmission, preventing infection, decreasing the likelihood of infection, decreasing the severity of infection, and/or decreasing duration of infection. The methods comprise administering one or more of the described combinations and formulations to a subject that is infected with a SARS-CoV-related betacoronavirus, suspected of being infected with a SARS-CoV-related betacoronavirus, or at risk of being infected with a SARS-CoV-related betacoronavirus.

In some embodiments, the described combinations and formulations can be used to inhibit SARS-CoV-related betacoronavirus entry into ACE2-expressing host cells, such as, but not limited to, human airway epithelia cells. In some embodiments, the described combinations and formulations can be used to inhibit SARS-CoV-2 entry into ACE2-expressing host cells, such as, but not limited to, human airway epithelia cells.

Described are methods of disrupting SARS-CoV-related betacoronavirus interaction with ACE2 in a subject. Disrupting interaction of SARS-CoV-related betacoronavirus with ACE2 inhibits viral entry and decreases viral transmission and disease burden. In some embodiments, the method comprise administering any of the described combinations or formulations to the subject. In some embodiments, the methods comprise administering any of the described combinations or formulations to the subject to inhibit SARS-CoV-related betacoronavirus entry into human airway cells.

Described are methods of disrupting SARS-CoV-2 interaction with ACE2 in a subject. Disrupting interaction of SARS-CoV-2 with ACE2 inhibits viral entry and decreases viral transmission and disease burden. In some embodiments, the methods comprise administering any of the described combinations or formulations to the subject. In some embodiments, the methods comprise administering any of the described combinations or formulations to the subject to inhibit SARS-CoV-2 entry into human airway cells.

In some embodiments, the diphenhydramine and lactoferrin are administered to a subject at dosage levels recognized or recommended for treating other conditions.

In some embodiments, the described diphenhydramine and lactoferrin are administered according to their recognized administration routes.

Diphenhydramine can be, but is not limited to, diphenhydramine citrate or diphenhydramine hydrochloride. Diphenhydramine can be provided as a liquid formulation, as a tablet, as a coated tablet, as a chewable tablet, as a powder, or as a capsule. Diphenhydramine can be administered orally, by inhalation (e.g., nasally), or parenterally. Parenteral administration can be, but is not limited to, intramuscular administration and intravenous administration. In some embodiments, diphenhydramine is administered orally. In some embodiments, diphenhydramine is administered by inhalation. In some embodiments, diphenhydramine is administered orally in water or phosphate buffered saline at about pH 7.4. In some embodiments, diphenhydramine is administered parenterally.

In some embodiments, an effective amount of diphenhydramine is about 5-600 mg, about 38-468 mg, about 25-402 mg, or about 25-300 mg. In some embodiments, an effective amount of diphenhydramine is up to about 228 mg/day, up to about 300 mg/day, up to about 400 mg/day, or up to about 456 mg/day. In some embodiments, an effective amount of diphenhydramine is about 10-100 mg, about 38-78 mg about 25-67 mg, or about 25-50 mg, administered orally every 4-6 hours. In some embodiments, an effective amount of diphenhydramine is about 10-100 mg or about 10-50 mg administered parenterally. In some embodiments, the diphenhydramine is administered 1, 2, 3, 4, 5, or 6 times per day.

In some embodiments, an effective amount of diphenhydramine is about 10-50 mg or about 12.5-25 mg administered 3-4 times/day. In some embodiments, an effective amount of diphenhydramine is about 10 mg, about 12.5 mg, about 25 mg or about 50 mg administered 3-4 times/day. In some embodiments, an effective amount of diphenhydramine is about 5 mg/kg. In some embodiments, an effective amount of diphenhydramine is about 150 mg/m².

In some embodiments, an effective amount of diphenhydramine is about 19-38 mg every 4-6 hours. In some embodiments, an effective amount of diphenhydramine is about 19 mg or about 38 mg every 4-6 hours. In some embodiments, an effective amount of diphenhydramine is about 38-76 mg every 4-6 hours. In some embodiments, an effective amount of diphenhydramine is about 38 mg or about 76 mg every 4-6 hours. In some embodiments, an effective amount of diphenhydramine is about 6.25 mg every 4-6. In some embodiments, an effective amount of diphenhydramine is about 12.5-25 mg every 4-6 h. In some embodiments, an effective amount of diphenhydramine is about 12.5 mg or about 25 mg every 4-6 h. In some embodiments, an effective amount of diphenhydramine is about 25-50 mg every 4-6 hours. In some embodiments, an effective amount of diphenhydramine is about 25 mg or about 50 mg every 4-6 hours. In some embodiments, an effective amount of diphenhydramine is about 1.25 mg/kg administered up to 4 times per day. In some embodiments, an effective amount of diphenhydramine is about 37.5 mg/m2 administered up to 4 times/day.

Lactoferrin can be, but is not limited to, unsaturated iron lactoferrin (apolactoferrin), hololactoferrin, recombinant lactoferrin or a fragment of lactoferrin having antiviral activity. Recombinant lactoferrin can be made from a plant, such a rice, from a microorganism, such as yeast or bacteria, or from mammalian or insect cells grown in culture. The lactoferrin can be, but is not limited to, human lactoferrin or bovine lactoferrin. The lactoferrin can be derived from or obtained from milk or colostrum. Lactoferrin can be provided as a liquid formulation, as a tablet, as a coated tablet, as a chewable tablet, as a powder, or as a capsule. Lactoferrin can be administered orally, by inhalation (e.g., nasally) or parenterally. Parenteral administration can be, but is not limited to, intramuscular administration and intravenous administration. In some embodiments, lactoferrin is administered orally. In some embodiments, lactoferrin is administered by inhalation. In some embodiments, diphenhydramine is administered parenterally.

In some embodiments, an effective amount of lactoferrin is about 100-5000 mg, about 250-4000 mg, about 500-3600 mg, about 1000-3600 mg, or about 1800-3600 mg. In some embodiments, an effective amount of lactoferrin is up about 250 mg/day, up to about 500 mg/day, up to about 1000 mg/day, up to about 1800 mg/day, or up to about 3600 mg/day. In some embodiments, an effective amount of lactoferrin is about 250 mg/day, about 500 mg/day, about 1000 mg/day, about 1500 mg/day, about 1800 mg/day, about 1900 mg/day, about 2000 mg/day, about 2100 mg/day, about 2200 mg/day, about 2300 mg/day, about 2400 mg/day, about 2500 mg/day, about 2600 mg/day, about 2700 mg/day, about 2800 mg/day, about 2900 mg/day, about 3000 mg/day, about 3100 mg/day, about 3200 mg/day, about 3300 mg/day, about 3400 mg/day, about 3500 mg/day, or about 3600 mg/day. In some embodiments, the lactoferrin is administered 1, 2, 3, 4, 5, or 6 times per day.

The diphenhydramine and lactoferrin are formulated for administration in vivo. The diphenhydramine and lactoferrin can be formulated together or for administration separately. In some embodiments, the diphenhydramine and lactoferrin are formulated together.

Diphenhydramine and lactoferrin can be provided together in a liquid formulation, a tablet, a coated tablet, a chewable tablet, a powder, or a capsule. Diphenhydramine and lactoferrin can be administered together orally, by inhalation (e.g., nasally), or parenterally. Parenteral administration can be, but is not limited to, intramuscular administration and intravenous administration. In some embodiments, diphenhydramine and lactoferrin administered together orally. In some embodiments, diphenhydramine and lactoferrin administered together by inhalation. In some embodiments, diphenhydramine and lactoferrin are administered together parenterally.

In some embodiments, the diphenhydramine and/or lactoferrin are formulated with one or more pharmaceutically acceptable excipients (including vehicles, carriers, diluents, and/or delivery polymers), thereby forming a pharmaceutical composition or medicament suitable for in vivo delivery to a subject, such as a human. The phrase “pharmaceutically acceptable” indicates that the substance or composition must be compatible chemically and/or toxicologically, with the other ingredients comprising a formulation, and/or the mammal being treated therewith.

A pharmaceutical composition or medicament includes a pharmacologically effective amount of diphenhydramine and/or lactoferrin and optionally one or more pharmaceutically acceptable excipients. Pharmaceutically acceptable excipients (excipients) are substances other than the Active Pharmaceutical ingredient (API, therapeutic product) that are intentionally included in the drug delivery system. Excipients do not exert or are not intended to exert a therapeutic effect at the intended dosage. Excipients may act to a) aid in processing of the drug delivery system during manufacture, b) protect, support or enhance stability, bioavailability or patient acceptability of the API, c) assist in product identification, and/or d) enhance any other attribute of the overall safety, effectiveness, of delivery of the API during storage or use. A pharmaceutically acceptable excipient may or may not be an inert substance.

Excipients include, but are not limited to: absorption enhancers, anti-adherents, anti-foaming agents, anti-oxidants, binders, buffering agents, carriers, coating agents, colors, delivery enhancers, delivery polymers, dextran, dextrose, diluents, disintegrants, emulsifiers, extenders, fillers, flavors, glidants, humectants, lubricants, oils, polymers, preservatives, saline, salts, solvents, sugars, suspending agents, sustained release matrices, sweeteners, thickening agents, tonicity agents, vehicles, water-repelling agents, and wetting agents.

The pharmaceutical compositions can contain other additional components commonly found in pharmaceutical compositions. Such additional components can include, but are not limited to: anti-pruritics, astringents, local anesthetics, or anti-inflammatory agents (e.g., antihistamine, diphenhydramine, etc.).

A carrier can be, but is not limited to, a solvent or dispersion medium containing, for example, water, ethanol, polyol (for example, glycerol, propylene glycol, and liquid polyethylene glycol), and suitable mixtures thereof. A carrier may also contain adjuvants such as preservatives, wetting agents, emulsifying agents and dispersing agents. A carrier may also contain isotonic agents, such as sugars, polyalcohols, sodium chloride, and the like into the compositions.

Pharmaceutically acceptable refers to those properties and/or substances which are acceptable to the subject from a pharmacological/toxicological point of view. The phrase pharmaceutically acceptable refers to molecular entities, compositions, and properties that are physiologically tolerable and do not typically produce an allergic or other untoward or toxic reaction when administered to a subject. In some embodiments, a pharmaceutically acceptable compound is approved by a regulatory agency of the Federal or a state government or listed in the U.S. Pharmacopeia or other generally recognized pharmacopeia for use in animals and more particularly in humans.

In some embodiments, the pharmaceutical compositions further comprise one or more additional active ingredients. The additional active ingredient can be, but is not limited to, an additional antiviral therapeutic, a pain reliver, or a nasal decongestant. In some embodiments, the additional active ingredient comprises an additional antiviral therapeutic. In some embodiments, the additional active ingredient comprises a pain reliever. The pain reliever can be, but is not limited to, acetaminophen, NSAID, ibuprofen, or naproxen. In some embodiments, the pain reliever is acetaminophen. The amount of acetaminophen in the formulation can be about 325 to about 1000 mg. In some embodiments, the additional active ingredient comprises a nasal decongestant. The nasal decongestant can be, but is not limited to, phenylephrine and pseudoephedrine.

The pharmaceutical composition can be in a liquid formulation, a tablet, a coated tablet, a chewable tablet, a powder (e.g., a lyophilized powder), or a capsule. The pharmaceutical composition can be administered orally, by inhalation, (e.g., nasally) or parenterally. Parenteral administration can be, but is not limited to, intramuscular administration and intravenous administration. In some embodiments, the pharmaceutical composition is administered orally. In some embodiments, the pharmaceutical composition is administered by inhalation. In some embodiments, the pharmaceutical composition is administered parenterally.

In some embodiments, the described pharmaceutical compositions are used for treating or managing clinical presentations associated with SARS-CoV-related betacoronavirus. In some embodiments, a therapeutically or prophylactically effective amount of diphenhydramine and lactoferrin are administered to a subject in need of such treatment, prevention or management. In some embodiments, administration of diphenhydramine and lactoferrin can be used to decrease the number, severity, and/or frequency of symptoms of a disease in a subject.

The described pharmaceutical compositions can be used to treat at least one symptom associated with SARS-CoV-related betacoronavirus in a subject. In some embodiments, the subject is administered a therapeutically effective amount of diphenhydramine and lactoferrin, thereby treating the symptom. In some embodiments, the subject is administered a prophylactically effective amount of one diphenhydramine and lactoferrin thereby preventing infection by a SARS-CoV-related betacoronavirus or preventing development of one or more symptoms associated with SARS-CoV-related betacoronavirus infection, such as COVID-19.

Symptoms associated with SARS-CoV-related betacoronavirus infection can be, but are not limited to, an inflammatory response, a cytokine storm, an inflammasome-associate response, an IL-1β-associated response, an NLRP3-associated response, lung fibrosis, pulmonary fibrosis, ground glass opacities, pulmonary fibrosis, acute respiratory distress syndrome (ARDS), pneumonia, and combinations thereof.

In some embodiments, the described pharmaceutical compositions can be administered to improve mucociliary transport or mitigate airway obstruction in a subject infected with a SARS-related betacoronavirus or suspected of being infected with a SARS-related betacoronavirus.

Described are methods of reducing the duration of SARS-CoV-2 infection, reducing the severity of SARS-CoV-2 infection, reducing the likelihood of SARS-CoV-2 infection, reducing SARS-CoV-2, or reducing the likelihood of developing COVID-19 in a subject, the method comprising administered to the subject any of the described combinations or formulations comprising diphenhydramine and lactoferrin.

In some embodiments, the described pharmaceutical compositions are administered to a subject at risk of infection by SARS-CoV-2, a subject that has tested positive for SARS-CoV-2, a subject that has been exposed to SARS-CoV-2, a subject suspected of having been exposed to SARS-CoV-2, a subject at risk of being exposed to SARS-CoV-2, a subject suffering from or diagnosed with COVID-19, or a subject suffering from acute lung injury due to SARS-CoV-2infection.

Described are methods of reducing the duration of SARS-CoV-related betacoronavirus infection, reducing the severity of SARS-CoV-related betacoronavirus infection, reducing the likelihood of SARS-CoV-related betacoronavirus infection, reducing SARS-CoV-related betacoronavirus replication, or reducing the likelihood of developing a SARS-CoV-related betacoronavirus-related illness in a subject, the methods comprising administering to the subject any of the described combinations or formulations of diphenhydramine and lactoferrin (e.g., any of the described pharmaceutical compositions). The SARS-CoV-related betacoronavirus can be, but is not limited to, SARS-CoV-2. The methods comprise administration of a therapeutically effective amount of the combinations, formulations, or pharmaceutical compositions as described herein to a subject, e.g., a human or animal subject in need of such treatment.

The combinations or formulations of diphenhydramine and lactoferrin can be administered to a subject at risk of infection by a SARS-CoV-related betacoronavirus, a subject that has tested positive for a SARS-CoV-related betacoronavirus, a subject that has been exposed to a SARS-CoV-related betacoronavirus, a subject suspected of having been exposed to a SARS-CoV-related betacoronavirus, a subject at risk of being exposed to a SARS-CoV-related betacoronavirus, or a subject suffering from or diagnosed with SARS-CoV-related betacoronavirus illness. The SARS-CoV-related betacoronavirus can be, but is not limited to, SARS-CoV-2.

It is to be understood that the disclosures are not to be limited to the specific embodiments disclosed and that modifications and other embodiments are intended to be included within the scope of the appended claims. The skilled artisan will recognize many variants and adaptations of the aspects described herein. These variants and adaptations are intended to be included in the teachings of this disclosure and to be encompassed by the claims herein.

### EXAMPLES

### Example 1. Hololactoferrin and Unsaturated Iron Lactoferrin (Apolactoferrin) have Anti-SARS-CoV-2 Activity

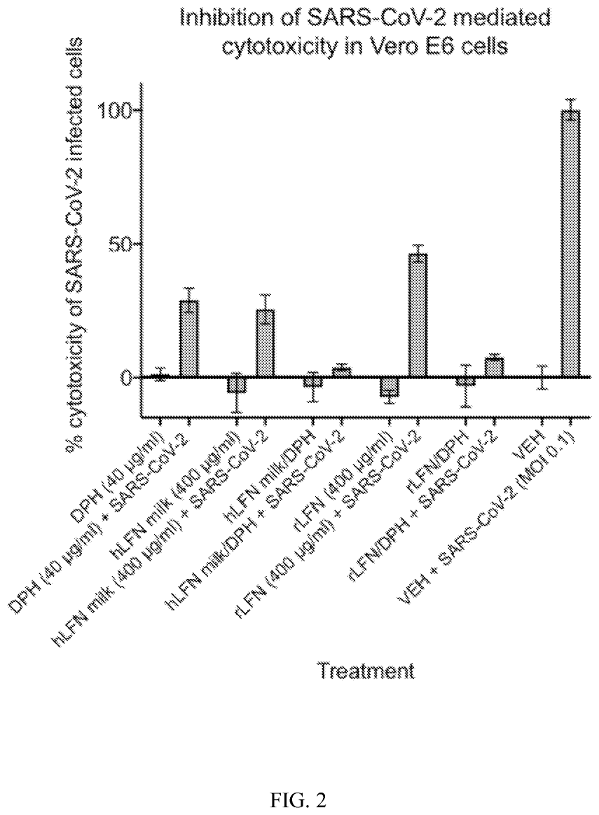

Hololactoferrin and unsaturated iron lactoferrin (apolactoferrin) were analyzed for direct antiviral activity against a SARS-CoV-2 isolate in vitro. Unsaturated iron lactoferrin directly inhibited SARS-CoV-2 infection more effectively compared to hololactoferrin. Results are shown in FIG. 2.

### Example 2. Diphenhydramine in Combination with Lactoferrin

Combinations of diphenhydramine and lactoferrin were tested in vitro to determine the antiviral effects of the combination against SARS-CoV-2. Hololactoferrin and unsaturated iron lactoferrin were used in combination with diphenhydramine. Unsaturated iron lactoferrin and diphenhydramine both inhibited SARS-CoV-2 infection when administered alone. When combined, diphenhydramine and unsaturated iron lactoferrin effectively inhibited virus infection. Further, the combination of diphenhydramine and unsaturated iron lactoferrin had a synergistic effect (i.e., more antiviral activity compared to additive effects of drugs administered singly) on inhibition of SARS-CoV-2 infection.

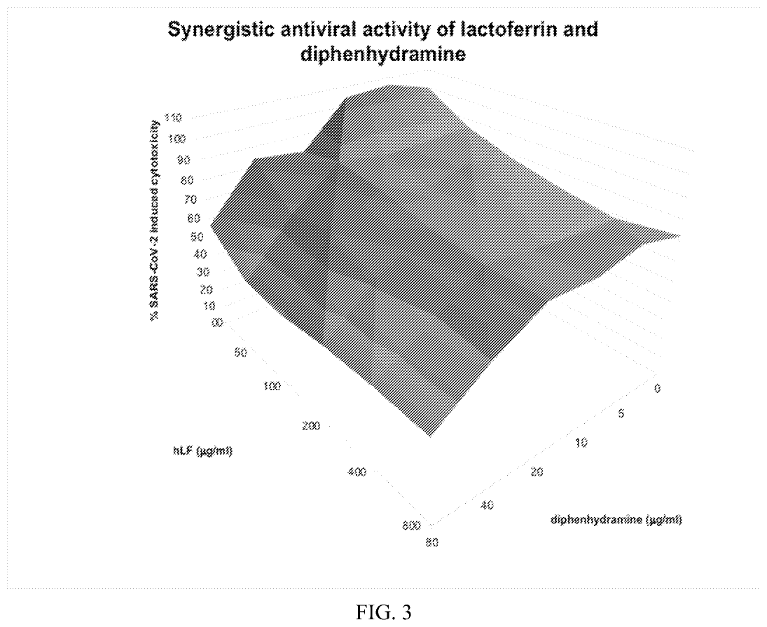

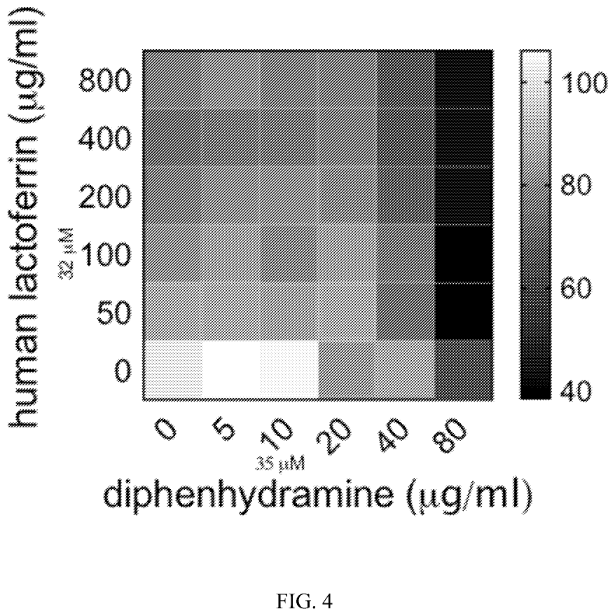

A) Checkerboard assay: Cells in incubated with varying formulations of diphenhydramine, unsaturated iron lactoferrin and recombinant iron saturated lactoferrin and exposed to SARS-CoV-2 at a MOI of 0.3. Lactoferrin was used at 0, 50, 100, 200, 400, or 800 μg/mL. Diphenhydramine was used at 0, 5, 10, 20, 40, or 80 μg/mL. Toxicity of SARS-CoV-2 was then determined. The data are shown in FIG. 2, FIG. 3, and FIG. 4. All treatments were effective at reducing virus-induced cytotoxicity. Unsaturated iron lactoferrin was more effective than recombinant lactoferrin. Both forms of lactoferrin exhibited a synergistic effect with diphenhydramine in reducing SARS-CoV-2 cytotoxicity (FIG. 2). FIG. 3 and FIG. 4 illustrate the synergistic nature DPH and LFN have on reduction of SARS-CoV-2 induced cytotoxicity.

As shown in FIG. 4, 5 μg/mL of DPH was unable to decrease SARS-CoV-2 induced cytotoxicity. The in vitro EC₅₀ diphenhydramine was 17.4 μg/mL. However, when 50 μg/mL of unsaturated iron lactoferrin was co-administered with 5 μg/mL of DPH, a significant reduction in SARS-CoV-2 induced cytotoxicity was observed, indicating the EC₅₀ of diphenhydramine is unsaturated when co-administered with unsaturated iron lactoferrin. The co-dependent dose effect can be seen in the trending of darker colors towards the upper right of FIG. 4.

Determination of Hill coefficient and EC₅₀.

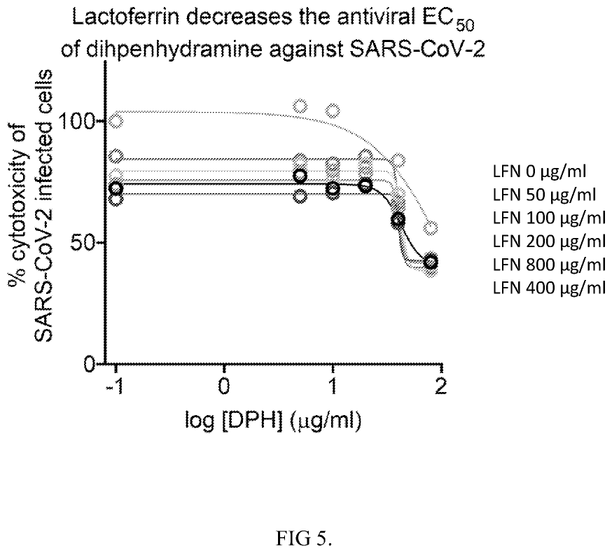

Percent cytotoxicity of SARS-CoV-2 infected cells was determined for varying concentrations of diphenhydramine and lactoferrin. Effective concentration 50 (EC50) curves illustrating the dose dependent inhibition of the SARS-CoV-2 induced cytotoxicity of diphenhydramine are shown in FIG. 5. In the presence of varying concentrations of unsaturated human lactoferrin, baseline cytotoxic inhibition and inhibitory velocity of SARS-CoV-2 induced cytotoxicity by diphenhydramine was greatly increased.

A Hill equation used to fit the data points and extrapolate the Hill coefficients associated with EC₅₀ curves exemplified in FIG. 5. A highly negative Hill-slope indicates increased inhibitory velocity upon SARS-CoV-2 near the EC₅₀.

Hill Slope Calculations: Y=1001(1+10^{((logIC50-X)*Hill slope)})

DPH+0 μg/ml LFN: Y=100/(1+10^{42-190-X)*-1.198)})

DPH+50 μg/ml LFN: Y=100/(1+10^{(0.611-X)*-20.75)})

DPH+100 μg/ml LFN: Y=100/(1+10^{((1.636-X)*-15.08)})

DPH+200 μg/ml LFN: Y=100/(1+10^{((1.613-X)*-16.36)})

DPH+400 μg/ml LFN: Y=100/(1+10^{((1.607-X)*-unstable)})

DPH+800 μg/ml LFN: Y=1001(1+10^{((1.631-X)*-4.714)})

EC₅₀ values extrapolated from FIG. 5. Percent cytotoxicity was as measured by cytotoxicity following 72 h infection of Vero E6 cells with SARS-CoV-2 at an MOI of 0.3:1. EC₅₀ of diphenhydramine in the absence of lactoferrin and in the presence of increasing lactoferrin concentrations demonstrated that the presence of LFN reduces the diphenhydramine EC₅₀ by 3 times as determined by cytotoxicity assays.

TABLE 1
EC₅₀ of Diphenhydramine in combination with indicated
levels of human unsaturated lactoferrin.

| | Lactoferrin (μg/mL) | | | | | |
|---|---|---|---|---|---|---|
| | 800 | 400 | 200 | 100 | 50 | 0 |
| Diphenhydramine | 42.76 | 40.44 | 41.01 | 43.28 | 40.79 | 155.1 |
| EC₅₀ (μg/mL) | | | | | | |

Thus, when a low concentration of lactoferrin is added in combination with diphenhydramine it greatly decreases the negative Hill slope while also decreasing the EC₅₀. This indicates a synergistic action in both potency and efficacy against SARS-CoV-2 induced cytotoxicity when applied in combination.

### Example 3. Combination Therapy for Prevention and/or Treatment of COVID-19 (SARS-CoV-2 Infection)

Based on the dosages of diphenhydramine and lactoferrin, when administered for treatment of other conditions, and based on the anti-SARS-COV-2 activity observed, we propose formulations of diphenhydramine HCl and Unsaturated iron lactoferrin for prevention and/or treatment of COVID-19 (SARS-CoV-2 infection).

In some embodiments, the formulation comprises 12.5-50 mg of diphenhydramine HCl and 1.8-3.6 g of unsaturated iron lactoferrin.

In some embodiments, the formulation comprises 12.5, 15, 20, 25, 30, 35, 40, 45, or 50 mg of diphenhydramine HCl and 1.8, 1.9, 2, 2.1, 2.2, 2.3, 2.4, 2.5, 2.6, 2.7, 2.8, 2.9, 3, 3.1, 3.1, 3.3, 3.5, 3.5, or 3.6 g of unsaturated iron lactoferrin

TABLE 2
Exemplary formulations for use in
treating or preventing COVID-19.

| | Diphenhydramine | unsaturated iron lactoferrin |
|---|---|---|
| Formulation | (mg) | (g) |
| 1 | 12.5 | 1.8 |
| 2 | 12.5 | 2.7 |
| 3 | 12.5 | 3.6 |
| 4 | 25 | 1.8 |
| 5 | 25 | 2.7 |
| 6 | 25 | 3.6 |
| 7 | 50 | 1.8 |
| 8 | 50 | 2.7 |
| 9 | 50 | 3.6 |

In some embodiments, the formulation further comprises a pain reliever. The pain reliever can be, but is not limited to, acetaminophen, NSAID, ibuprofen, naproxen. In some embodiments, the pain reliever is acetaminophen. Acetaminophen can be added to any of the above listed formations. The amount of acetaminophen in the formulation can be about 325 to about 1000 mg

In some embodiments, the formulation further comprises a nasal decongestant. The nasal decongestant can be, but is not limited to, phenylephrine and pseudoephedrine.

TABLE 3
Exemplary formulations for use in
treating or preventing COVID-19.

| Formulation | antihistamine | Co-therapeutic |
|---|---|---|
| 10 | Diphenhydramine | unsaturated iron lactoferrin |
| 11 | Diphenhydramine | Acetaminophen |
| 12 | Diphenhydramine | Phenylephrine |
| 13 | Diphenhydramine | dextromethorphan HBr |
| 14 | Diphenhydramine | Bismuth subsalicylate |
| 15 | Diphenhydramine | Loperamide |
| 16 | Diphenhydramine | Famotidine |
| 17 | Hydroxyzine | unsaturated iron lactoferrin |
| 18 | Hydroxyzine | Acetaminophen |
| 19 | Hydroxyzine | Phenylephrine |
| 20 | Hydroxyzine | dextromethorphan HBr |
| 21 | Hydroxyzine | Bismuth subsalicylate |
| 22 | Hydroxyzine | Loperamide |
| 23 | Hydroxyzine | Famotidine |
| 24 | Cetirizine | unsaturated iron lactoferrin |
| 25 | Cetirizine | Acetaminophen |
| 26 | Cetirizine | Phenylephrine |
| 27 | Cetirizine | dextromethorphan HBr |
| 28 | Cetirizine | Bismuth subsalicylate |
| 29 | Cetirizine | Loperamide |
| 30 | Cetirizine | Famotidine |
| 31 | Azelastine | unsaturated iron lactoferrin |
| 32 | Azelastine | Acetaminophen |
| 33 | Azelastine | Phenylephrine |
| 34 | Azelastine | dextromethorphan HBr |
| 35 | Azelastine | Bismuth subsalicylate |
| 36 | Azelastine | Loperamide |
| 37 | Azelastine | Famotidine |
| 38 | Loratadine | unsaturated iron lactoferrin |
| 39 | Loratadine | Acetaminophen |
| 40 | Loratadine | Phenylephrine |
| 41 | Loratadine | dextromethorphan HBr |
| 42 | Loratadine | Bismuth subsalicylate |
| 43 | Loratadine | Loperamide |
| 44 | Loratadine | Famotidine |
| 45 | Levocetirizine | unsaturated iron lactoferrin |
| 46 | Levocetirizine | Acetaminophen |
| 47 | Levocetirizine | Phenylephrine |
| 48 | Levocetirizine | dextromethorphan HBr |
| 49 | Levocetirizine | Bismuth subsalicylate |
| 50 | Levocetirizine | Loperamide |
| 51 | Levocetirizine | Famotidine |
| 52 | Brompheniramine | unsaturated iron lactoferrin |
| 53 | Brompheniramine | Acetaminophen |
| 54 | Brompheniramine | Phenylephrine |
| 55 | Brompheniramine | dextromethorphan HBr |
| 56 | Brompheniramine | Bismuth subsalicylate |
| 57 | Brompheniramine | Loperamide |
| 58 | Brompheniramine | Famotidine |
| 59 | Fexofenadine | unsaturated iron lactoferrin |
| 60 | Fexofenadine | Acetaminophen |
| 61 | Fexofenadine | Phenylephrine |
| 62 | Fexofenadine | dextromethorphan HBr |
| 63 | Fexofenadine | Bismuth subsalicylate |
| 64 | Fexofenadine | Loperamide |
| 65 | Fexofenadine | Famotidine |
| 66 | Chlorpheniramine | unsaturated iron lactoferrin |
| 67 | Chlorpheniramine | Acetaminophen |
| 68 | Chlorpheniramine | Phenylephrine |
| 69 | Chlorpheniramine | dextromethorphan HBr |
| 70 | Chlorpheniramine | Bismuth subsalicylate |
| 71 | Chlorpheniramine | Loperamide |
| 72 | Chlorpheniramine | Famotidine |

TABLE 4
Exemplary formulations for use in treating or preventing COVID-19.

| | H1 receptor | H2 receptor | | | | | |
|---|---|---|---|---|---|---|---|
| | blocking | blocking | | | | Anti-nausea/ | |
| Formulation | antihistamine | antihistamine | NSAID | Cough suppressant | Decongestant | Anti-diarrhea | Lactoferrin |
| 73 | Hydroxyzine | Famotidine | Acetaminophen | Dextromethorphan | Phenylephrine | Bismuth | Lactoferrin |
| | | | | HBr | HCl | subsalicylate | |
| 74 | Cetirizine | Famotidine | Acetaminophen | Dextromethorphan | Phenylephrine | Bismuth | Lactoferrin |
| | | | | HBr | HCl | subsalicylate | |
| 74 | Loratadine | Famotidine | Acetaminophen | Dextromethorphan | Phenylephrine | Bismuth | Lactoferrin |
| | | | | HBr | HCl | subsalicylate | |
| 76 | Diphenhydramine | Famotidine | Acetaminophen | Dextromethorphan | Phenylephrine | Bismuth | Lactoferrin |
| | HCl | | | HBr | HCl | subsalicylate | |
| 77 | Azelastine | Famotidine | Acetaminophen | Dextromethorphan | Phenylephrine | Bismuth | Lactoferrin |
| | | | | HBr | HCl | subsalicylate | |
| 78 | Hydroxyzine | Famotidine | Acetaminophen | Dextromethorphan | Phenylephrine | Loperamide | Lactoferrin |
| | | | | HBr | HCl | | |
| 79 | Cetirizine | Famotidine | Acetaminophen | Dextromethorphan | Phenylephrine | Loperamide | Lactoferrin |
| | | | | HBr | HCl | | |
| 80 | Loratadine | Famotidine | Acetaminophen | Dextromethorphan | Phenylephrine | Loperamide | Lactoferrin |
| | | | | HBr | HCl | | |
| 81 | Diphenhydramine | Famotidine | Acetaminophen | Dextromethorphan | Phenylephrine | Loperamide | Lactoferrin |
| | HCl | | | HBr | HCl | | |
| 82 | Azelastine | Famotidine | Acetaminophen | Dextromethorphan | Phenylephrine | Loperamide | Lactoferrin |
| | | | | HBr | HCl | | |

### Example 4. Synergistic Antiviral Activity by Combining a Sigma Receptor Ligand with Lactoferrin

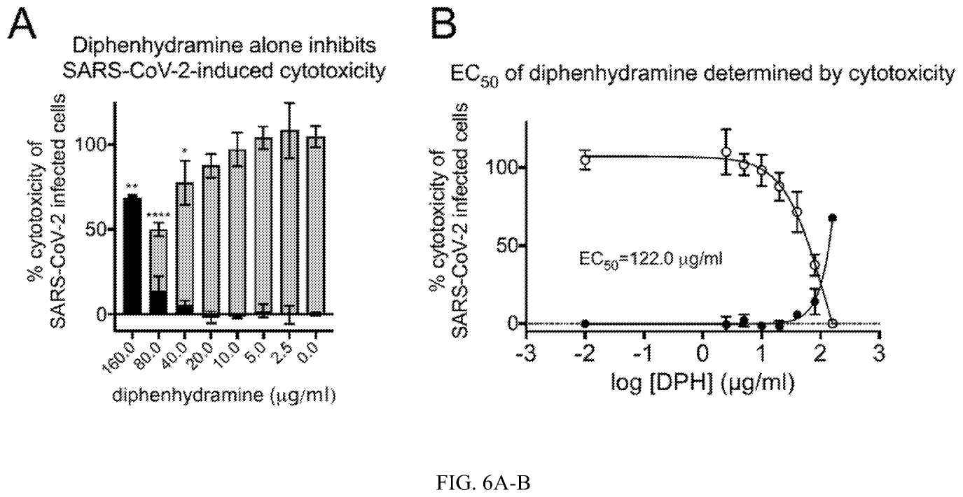

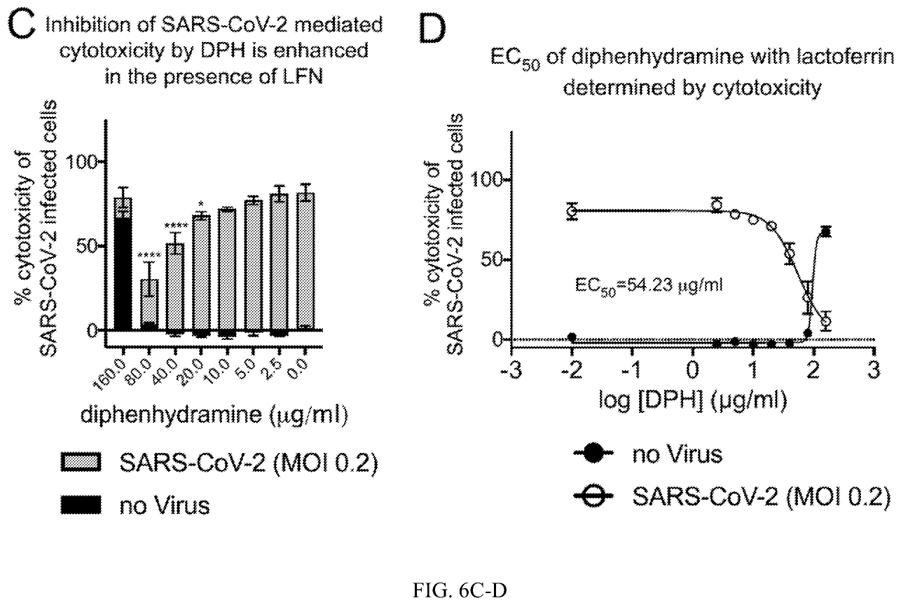

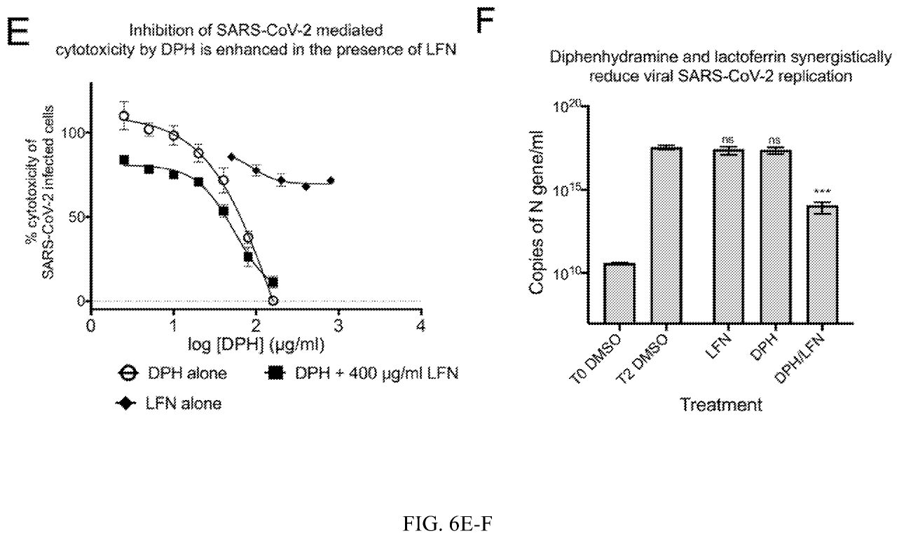

Diphenhydramine was recently shown to inhibit SARS-CoV-2 infectivity and the calculated EC₅₀ for SARS-CoV-2 by plaque reduction assay was 17.4 μg/ml (59.6 μM). This drug is safe, well-characterized, and widely available and so highly relevant in the search for COVID therapeutics. Additional studies showed the ability of diphenhydramine to inhibit SARS-CoV-2 induced cytotoxicity and found an EC₅₀ of 122.0 μg/ml (418 μM; FIGS. 6A and B), about 7 times higher than that found in the plaque reduction assay. We tested whether diphenhydramine could be combined with latoferrin to reduce its EC₅₀ for antiviral activity against SARS-CoV-2.

Diphenhydramine was combined with lactoferrin and tested for anti SARS-CoV-2 activity. The host-iron sequestration protein lactoferrin was reported to exhibit direct antiviral activity against SARS-CoV-2²⁸⁻²⁹, is broadly antimicrobial, and possesses host immunostimulatory properties. Various combinations of lactoferrin with diphenhydramine were analyzed to measure effects on reduction of EC₅₀. Co-administration of 400 μg/ml of lactoferrin with diphenhydramine further reduced SARS-CoV-2 induced cytotoxicity and decreased the EC₅₀ by 55.5% to 54.2 μg/ml (185.7 μM; FIGS. 6C and D). The antiviral enhancement effects of lactoferrin were more apparent at lower concentrations of diphenhydramine (FIG. 6E) Inhibition of viral replication was also investigated by qPCR (FIG. 6F). Combining lactoferrin with diphenhydramine resulted in synergistic effects on anti-viral activity against SARS-CoV-2. 40 μg/ml diphenhydramine alone resulted in 32.2% reduction in N-protein RNA compared to DMSO alone controls. 400 μg/ml lactoferrin was able to decrease N-protein RNA copies by 28.0% 48 h after infection. When combined, diphenhydramine and lactoferrin inhibited 99.97% of N-protein RNA copies, a 3-log reduction that was highly significant. These data demonstrate that diphenhydramine and lactoferrin, both with well characterized safety profiles, have synergistic effects on inhibition of SARS-CoV-2.

Virus culture methods. The SARS-CoV-2 strain used in this study was UF-1. It was isolated from a COVID19 patient at UF Health Shands Hospital via nasal swab. Vero E6 cells were grown in DMEM+2% FBS+PenStrep. SAEC, H23 and H23-hACE2 cells were grown in RPMI+10% FBS+PenStrep with 4 μg/ml of blasticidin to maintain ACE2 expression if needed. Cell were grown at 37° C. and 5% CO2 in a humidified incubator. An EVOS XL Core microscope was used to visualize cells.

Quantitation of virus replication by qPCR. SARS-CoV-2 was used to infect Vero E6 monolayers at an MOI of 0.01 in the presence of each treatment in biological and technical triplicate. At 2 days post-infection (dpi), the monolayers were scraped and harvested into viral lysis buffer (buffer AVL) from the QIAamp Viral RNA Kit (QIAGEN). The AVL buffer is a CDC approved method of viral inactivation. Samples were frozen at −80° C. RNA was purified according to the manufacturer's recommendations. Reverse transcription and cDNA synthesis was accomplished using the iTaq Universal SYBR Green One-Step Kit (BioRad) and primers targeting the nucleocapsid (N) gene of SARS-CoV-2 (NproteinF-GCCTCTTCTCGTTCCTCATCAC SEQ ID NO: 2, NproteinR-AGCAGCATCACCGCCATTG SEQ ID NO: 3). qPCR was carried out on a BioRad CFX96. N protein copy levels were calculated using CT values from a standard curve generated using a control plasmid containing the N protein gene and are presented as genome equivalents (GE) (Integrated DNA Technologies)

Cytotoxicity reduction assays. Vero E6 cells were seeded into 96-well CellBind treated plates (Corning) and allowed to attach overnight. Drugs were pre-aliquoted in DMEM+2% FBS. Titered SARS-CoV-2 aliquots were diluted to produce a target MOI of 0.2 PFU/cell in solution at the final indicated drug concentrations. Triplicate monolayers were infected by replacing growth media with 100 μl of the drug/virus suspensions. At 72 h post infection, supernatants were harvested, and lactate dehydrogenase (LDH) release was assayed using the Cytox 96™ Non-Radioactive Cytotoxicity Assay (Promega). Assays were performed as recommended by the manufacturer to generate a formazan dye. The optical density at 450 nm was measured using a MultiSkan FC plate reader (ThermoFisher). Controls included total LDH release as measured by lysis of all cells, spontaneous release from uninfected cells, and media alone. The toxicity of treatment alone was also determined in parallel to discriminate the amount of SARS-CoV-2-induced cytotoxicity occurring in the presence of a given treatment. After spontaneous and background subtraction, OD450 values were transformed to a percent of SARS-CoV-2 infected cells (100%) in the absence of any drug treatment to obtain percent of SARS-CoV-2-induced cytotoxicity. These experiments were carried out twice.

Inhibitory concentration and effective concentration calculations. CC₅₀ values and EC₅₀ values were calculated using the GraphPad Prism 9 software nonlinear regression module.

### Example 5. Diphenhydramine Plus Lactoferrin Inhibit Infectious Particle Production in Human Lung Cells

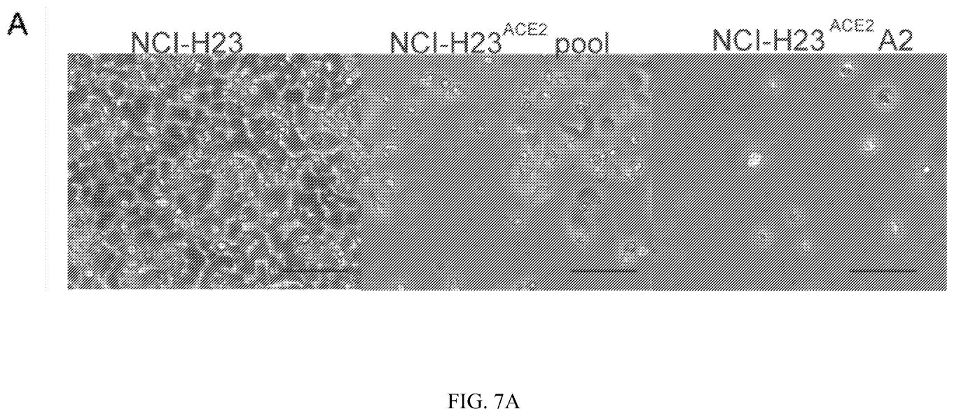

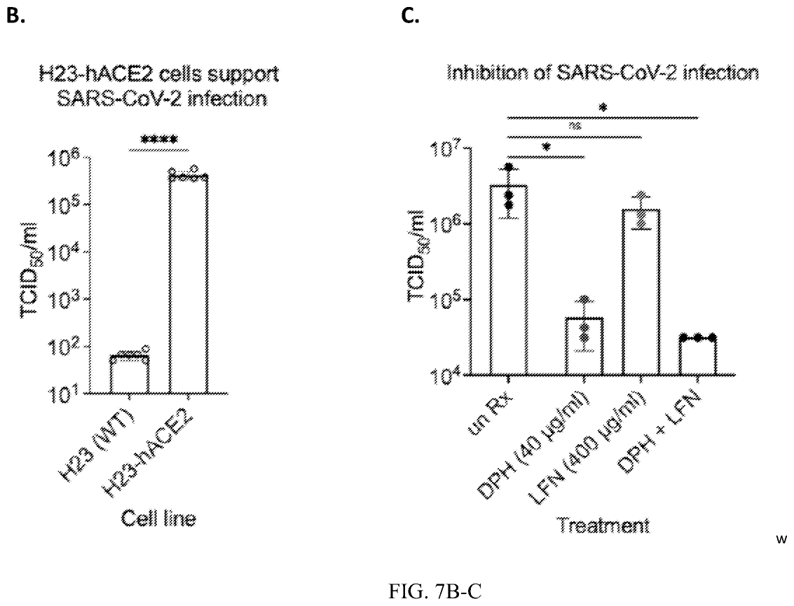

A new human lung epithelial cell line susceptible to SARS-CoV-2 infection, H23-ACE2, was generated by lentivirus transduction to introduce the human ACE2 gene. Single clone isolation of the H23-ACE2 transduced cell pool resulted in several healthy clones, including clone A2. Successful ACE2 expression was functionally indicated by increased cytopathic effect upon SARS-CoV-2 infection of an H23-ACE2 cell pool and an isolated cell clone H23-ACE2 clone A2 but not the parental H23 cell line (FIG. 7A). ACE2 surface expression was confirmed by flow cytometry as a peak shift to the right on the X-axis towards for H23-ACE2 cell pool and H23-ACE2 clone A2 compared to the untransduced parent H23 cell line and Vero E6 cells. H23-ACE2 clone A2 was used for further experiments. SARS-CoV-2 was used to infect the human lung epithelial cell line H23 at an MOI of 0.01. This cell line is unable to support SARS-CoV-2 infection without heterologous expression of the ACE-2 receptor. hACE2 expression was shown to be required for SARS-CoV-2 infection (FIGS. 7A and B). TCID50s were performed to measure infectious particles released during infection in the presence of diphenhydramine, lactoferrin and diphenhydramine+lactoferrin. Cells were originally infected at an MOI of 0.01 which is equivalent to about 1.5×10³ virus. The ability of diphenhydramine alone, lactoferring alone, and the combination in reducing SARS-CoV-2 release during infection in this cell line is shown in FIG. 7C.

Generation of ACE-2 lentivirus particles. The lentivirus containing ACE2 were generated by co-transfecting psPAX2, pMD2.G, and an ACE expression vector that also contained a blasticidin selection gene EX-U1285-Lv197 (GeneCopoeia). The plasmids were transfected into HEK293T cells using X-tremeGENE 9 (Roche Cat #XTG9-RO) as per the manufacturer's instructions. Media was replaced with DMEM containing 2% (w/v) bovine serum albumin (BSA) 18 h post transfection and then lentiviruses were collected after 24 and 48 h.

ACE2 transduction of NCI-H23 cells and monoclonal cell selection. NCI-H23 (aka H23) cells were obtained from ATCC (CRL-5800) and ACE2 lentiviruses were filtered through a 0.45 μm filter and used to transduce H23 cells using reverse transduction. Briefly, filtered virus particles are added to the H23 cell suspension with RPMI 1640 (Gibco Cat #1185093) media supplemented with 10% FBS and 8 μg/ml polybrene (Sigma Cat #TR-1003-G). 72 h post transduction, media was changed to RPMI 1640 supplemented with 10% FBS and 4 μg/ml blasticidin S hydrochloride (Gibco Cat #R21001). Cells were expanded in increasingly larger cell culture plates and ACE2 expression was confirmed by infecting with SARS-CoV-2 (strain Canada/ON/VIDO-01/2020) and flow cytometry. Single clone isolation from the H23-ACE2 cell pool was carried out by the array dilution method in 96-well plates. Single clones were collected 2-3 weeks after seeding and expanded in increasingly larger cell culture plates. After successful isolation, cells were maintained with complete media containing 2 μg/ml blasticidin.

Analysis of cell surface ACE2 by flow cytometry. Healthy cells were detached from the monolayer using 0.5 mM EDTA in PBS and centrifuged at 1500 rpm for 3 min. The cell pellet was stained for 1 h at 4° C. with primary ACE2 antibody (R&D systems Cat #AF933, used at a concentration of 0.25 μg/106 cells). The cells were then washed twice with flow wash buffer (2% FBS in PBS) and stained with secondary Goat IgG APC conjugated antibody (R&D systems Cat #F0108, at recommended volume of 10 W/106 cells), 1000× live-dead viability stain (Invitrogen Cat #L34958) and fixed with 2% PFA (diluted in flow wash buffer). The cells were analyzed using a Beckman CytoFLEX Flow Cytometer and the CytoExpert software.

TCID50 assays in H23 cells. H23 or H23-hACE2 cells were seeded at 1.5×105 cells in Corning CellBIND 24-well plates and allowed to attach overnight. The next day, SARS-CoV-2 was used to infect the cells at an MOI of 0.01 in the presence of mock treatment (PBS), diphenhydramine (40 μg/ml), lactoferrin from human milk (400 μg/ml), or a combination of diphenhydramine (40 μg/ml) and lactoferrin (400 μg/ml). The TCID50s were performed by diluting 48 h supernatant from the H23 infections across 8 columns of Vero E6 cells in three independent experiments. Five days later the TCID plates were observed by microscopy for CPE. TCID50/ml in the original H23 infection culture supernatant were calculated by the method of Spearman-Kärber. The TCID experiments were carried out in technical triplicate as described above with individual TCID50/ml values and their average and standard deviation shown.

1. A method of treating a subject suffering from infection by or susceptible to infection by a SARS-CoV-related betacoronavirus comprising administering to a subject a therapeutically effective amount of diphenhydramine and lactoferrin.

2. A method of treating a subject suffering from a SARS-CoV-related betacoronavirus-related illness comprising administering to the subject a therapeutically effective amount of diphenhydramine and lactoferrin.

3. A method of preventing infection by a SARS-CoV-related betacoronavirus comprising administering to a subject a therapeutically effective amount of diphenhydramine and lactoferrin.

4. The method of any one of claims 1-3, wherein the lactoferrin is a human lactoferrin or a bovine lactoferrin.

5. The method of any one of claims 1-4, wherein the method further comprises administering one or more additional active ingredients.

6. The method of claim 5, wherein the additional active ingredients are selected from the group consisting of: an additional antiviral therapeutic, a pain reliver, and a nasal decongestant.

7. The method of claim 6, wherein the pain reliver comprises acetaminophen.

8. The method of any one of claims 1-2 and 4-7, wherein the subject has tested positive for a SARS-CoV-related betacoronavirus, has been exposed to a SARS-CoV-related betacoronavirus, is suspected of having been exposed to a SARS-CoV-related betacoronavirus, is at risk of being exposed to a SARS-CoV-related betacoronavirus, is suffering from or diagnosed with a SARS-CoV-related betacoronavirus-related illness, or is suffering from acute lung injury due to a SARS-CoV-related betacoronavirus-related illness.

9. The method of any one of claims 1-8, wherein the SARS-CoV-related betacoronavirus is SARS-CoV-2.

10. The method of any one of claims 2 and 4-9, wherein the SARS-CoV-related betacoronavirus-related illness is COVID-19.

11. A pharmaceutical composition comprising diphenhydramine and lactoferrin.

12. The pharmaceutical composition of claim 11, further comprising a pharmaceutically acceptable excipient.

13. The pharmaceutical composition of claim 12, further comprising one or more additional active ingredients.

14. The pharmaceutical composition of claim 13, wherein the one or more additional active ingredients is/are selected from the group consisting of: an additional antiviral therapeutic, a pain reliver, and a nasal decongestant.

15. The pharmaceutical composition of any one of claims 11-14, wherein the pharmaceutical composition is formulated as liquid, a tablet, a coated tablet, a chewable tablet, a powder, capsule, or nasal formulation.

16. A pharmaceutical composition of any one of claims 11-15, for use in treating a SARS-CoV-related betacoronavirus infection or a SARS-CoV-related betacoronavirus-related disease.

17. The pharmaceutical composition of claim 16, wherein the SARS-CoV-related betacoronavirus is SARS-CoV-2.

18. The pharmaceutical composition of claim 16, wherein the SARS-CoV-related betacoronavirus-related disease is COVID-19.

19. A method of treating a subject suffering from infection by or susceptible to infection by a SARS-CoV-related betacoronavirus comprising administering to a subject a pharmaceutical composition comprising a therapeutically effective amount of H1 receptor blocking antihistamine and a therapeutically effective amount of lactoferrin.

20. The method of claim 19, wherein the H1 receptor blocking antihistamine is selected from the group consisting of: diphenhydramine, hydroxyzine, cetirizine, azelastine, loratadine, levocetirizine, brompheniramine, fexofenadine, and chlorpheniramine.

21. The method of claim 19 or 20, wherein the pharmaceutical composition further comprises a H2 receptor blocking antihistamine.

22. The method of claim 21, wherein the H2 receptor blocking antihistamine comprises famotidine.

23. The method of any one of claims 19-22, wherein the pharmaceutical composition further comprises a NSAID.

24. The method of claim 23, wherein the NSAID comprises Acetaminophen.

25. The method of any one of claims 19-24, wherein the pharmaceutical composition further comprises a cough suppressant.

26. The method of claim 25, wherein the cough suppressant comprises dextromethorphan.

27. The method of any one of claims 19-26, wherein the pharmaceutical composition further comprises a decongestant.

28. The method of claim 27, wherein the decongestant comprises phenylephrine.

29. The method of any one of claims 19-28, wherein the pharmaceutical composition further comprises an anti-nausea or anti-diarrhea medication.

30. The method of claim 29, wherein the anti-nausea or anti-diarrhea medication comprises bismuth subsalicylate or loperamide.
